(19) United States
(12) Patent Application Publication
(10) Pub. No.: US 2008/0172061 A1
(43) Pub. Date: Jul. 17, 2008

(54) Assembly Consisting of Insertion/Extraction Device and Part Used in Orthopaedic Surgery

(76) Inventor: Sheila Ragbir, Whim (TT)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/883,035

(22) PCT Filed: Jan. 25, 2006
(86) PCT No.: PCT/IB06/00113

§ 371 Date: Jul. 25, 2007

(30) Foreign Application Priority Data
Jan. 28, 2005 (CH) 00134/05
Dec. 30, 2005 (CH) 02100/05

(51) Int. Cl. A61B 17/58; A61B 17/00

(52) U.S. Cl. 606/99; 606/208

(57) ABSTRACT

An assembly consisting of an insertion/extraction device and a preparatory instrument or a prosthetic part used in orthopeadic surgery operations. The free end of the said preparatory instrument or that prosthetic part has, formed therein, a first recess able to be engaged in a complementary manner with the end of a modular neck, and a second recess having an axis substantially intersecting the axis of the first recess. The device is provided with a handle having its axis parallel to that of the second recess and having a first terminal portion shaped so as to engage in a complementary manner with the recess, and a second terminal portion adjacent to the first recess and connected to means which are fixed onto the handle and which, when they are operated, perform the coaxial insertion of the portion into the second recess.

The present invention relates to the sector of insertion/extraction devices used to perform orthopaedic surgical operations intended to implant replacement prostheses for articulations damaged by degenerative diseases, traumas, etc.

In order to perform these operations, it is known that surgeons must insert into the intramedullary channel of a bone (shoulder, thigh) preparatory instruments which form inside it a cavity of predefined shape and then insert a prosthesis into this cavity.

A good example of these preparatory instruments consists of a rasp, which has the same shape as the prosthesis to be inserted and which has a surface covered with tiny cutting edges which, when the said rasp is pushed and suitably manoeuvred, form the cavity with the desired shape by removing shavings and/or fragments.

An example of a prosthesis may instead be regarded as a stem which is inserted into the abovementioned cavity.

Both the preparatory instruments and the prosthetic parts must therefore be introduced with force inside the intramedullary channel and, especially the preparatory instruments, must also be extracted from it after a desired operation has been performed.

In the case of a rasp, for example, since it has the same shape as the prosthesis, and therefore is tapered, after carrying out the above operation, also owing to the presence of the said cutting edges, it remains to a certain extent attached to the bone inside which it has been inserted and, in order to extract it, pulling forces which are awkward and even potentially dangerous for the integrity of the bone must be applied.

Similar problems exist as regards the application and extraction, where necessary, of the stem of a prosthesis. It would therefore be necessary to be able to have a device able to allow easy and safe execution of the abovementioned operations.

The inventor of the subject of the present application has provided an innovative solution, devising a simple and functional assembly consisting of an insertion/extraction device and part used in orthopaedic surgery, which allows the insertion and extraction of a preparatory instrument and/or a prosthetic part to be performed in a careful and precise manner. In this assembly the handle has the characteristic feature that it may be joined integrally to instruments or prosthetic parts of different size and that it may be easily disengaged from them, leaving them inside the cavity formed, and may be joined to them again in order to be able to extract them.

The main characteristic feature of the abovementioned assembly consists in that the preparatory instrument or prosthesis to be applied have two recesses arranged alongside each other with substantially intersecting axes. Of them, one of them is able to contain the end of a modular neck of a prosthesis and the other one is a recess with a rectangular, preferably cylindrical shape. The insertion/extraction device according to the invention, which will be described more fully below, has two terminal portions with shapes matching those of the two said recesses, one of which may be made to slide parallel to the handle of the device, by operating means (levers, threaded elements, etc.) connected to it and integral with the handle itself.

If the terminal portion of the insertion/extraction device matching the shaped recess for receiving the end of a modular neck of a prosthesis is first applied and inserted inside it, and then the other terminal portion is made to slide by operating the said means and causing them to penetrate inside the said preferably cylindrical recess, owing to orientation of the axes of the two said recesses which intersect each other, gripping of the preparatory instrument or the prosthetic part between the abovementioned terminal portions is obtained, resulting in secure and reliable gripping thereof.

It is therefore possible to insert it and/or extract it into/from the bone by exerting respectively a simple pushing or pulling force on the abovementioned handle.

The subject of the present invention therefore consists of an assembly as described in the accompanying claim 1.

A more detailed description of two preferred embodiments of the assembly according to the invention will now be described, with reference also to the accompanying drawings in which:

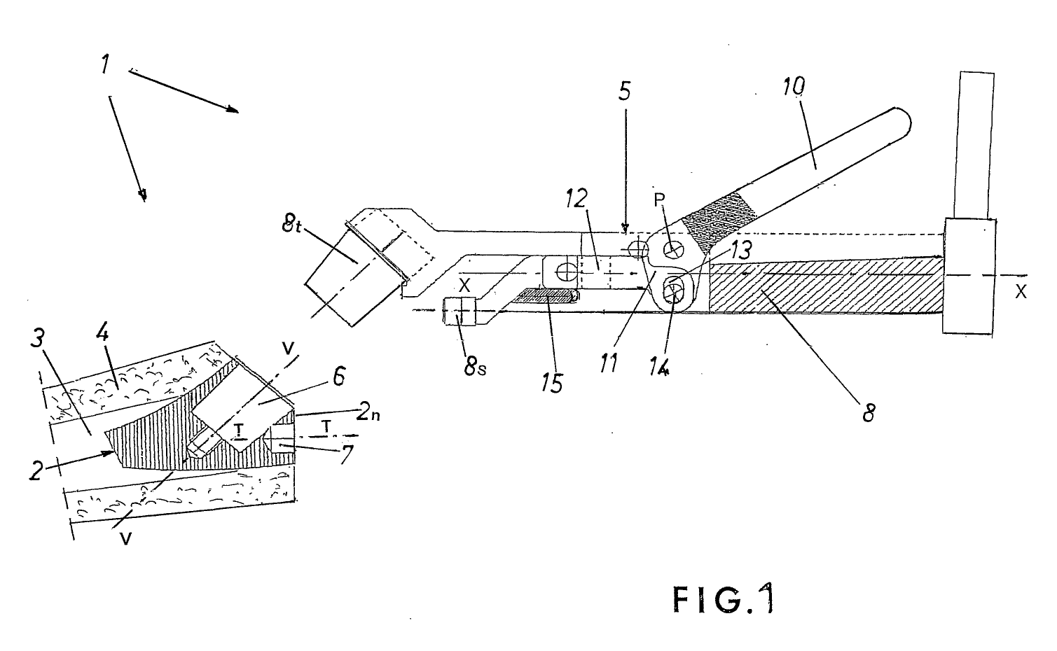

FIG. 1 is a partially sectioned side view of a first example of an assembly according to the invention with a rasp inserted inside a bone and the associated device at a distance therefrom.

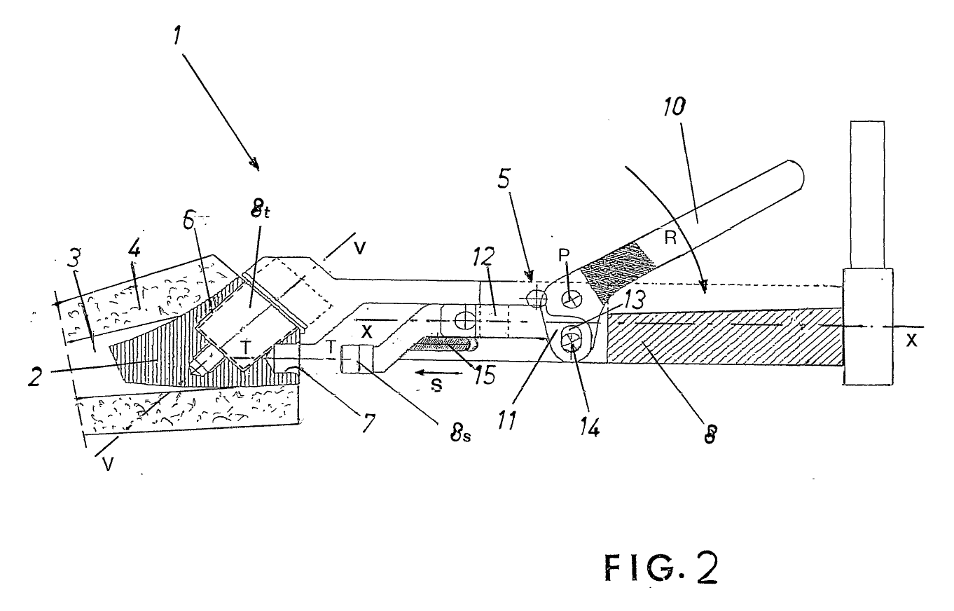

FIG. 2 is the same view as FIG. 1 with only the said terminal portion of the handle shaped like the end of a modular neck of a prosthesis inside the respective recess;

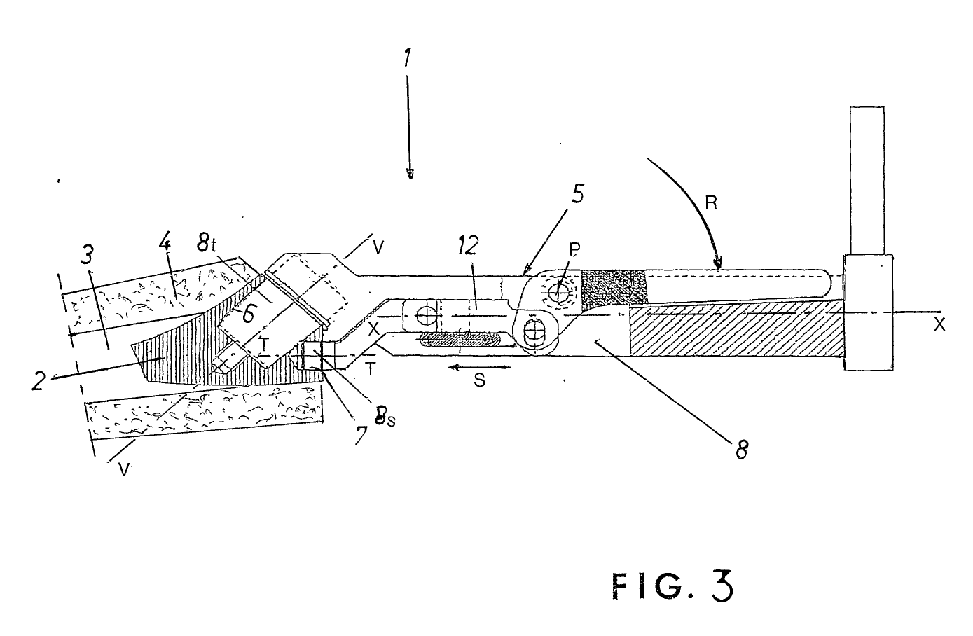

FIG. 3 is the same view as the preceding figures, but with both the terminal portions of the handle inserted inside the respective recesses.

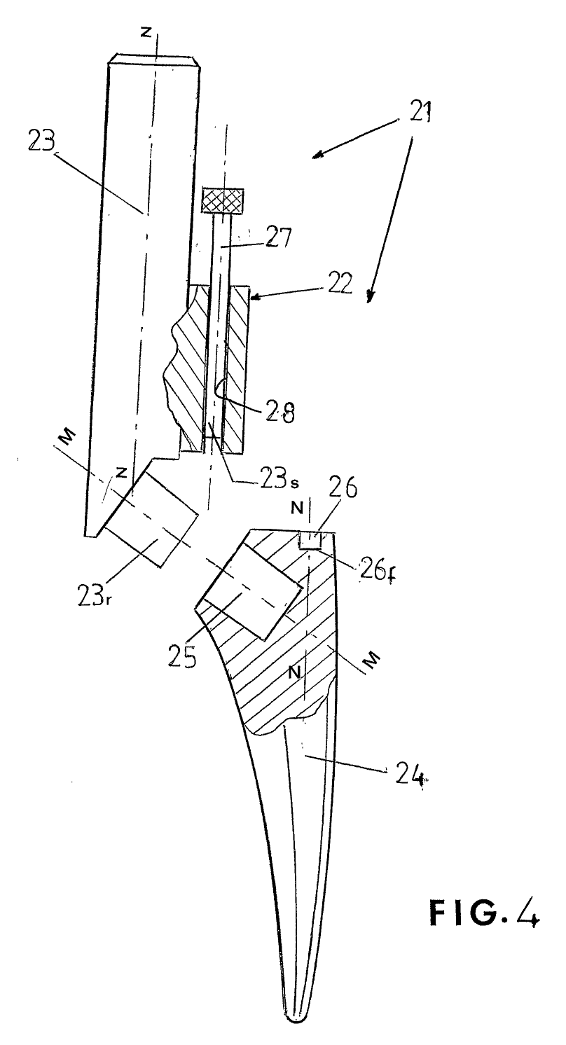

FIG. 4 is a longitudinal section through a second example of an insertion/extraction device with a stem forming part of a prosthesis situated at a distance therefrom.

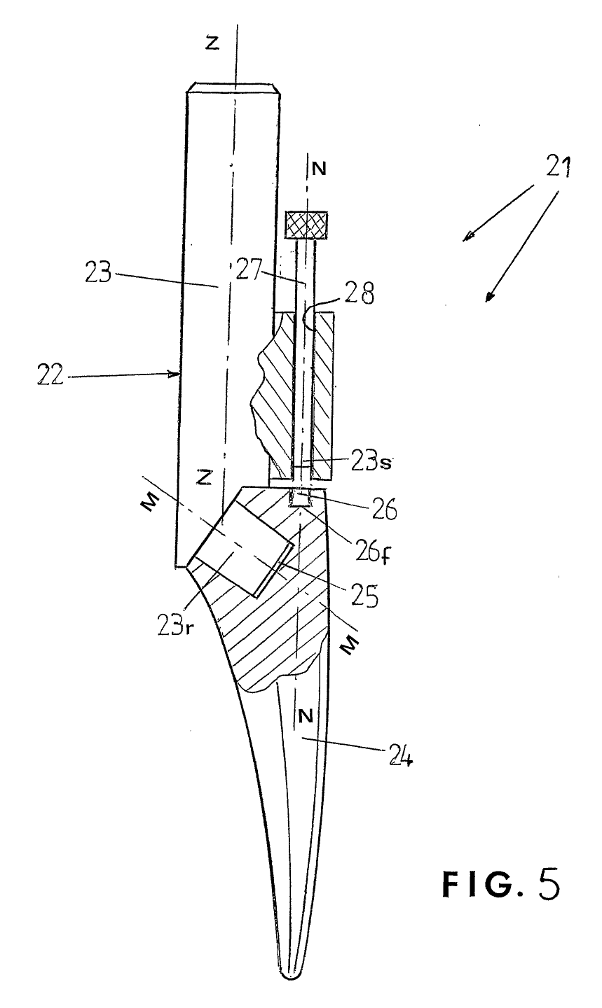

FIG. 5 is a longitudinal section through the device according to FIG. 4 with a terminal portion engaged in a complementary manner inside one of the two recesses formed in the said stem;

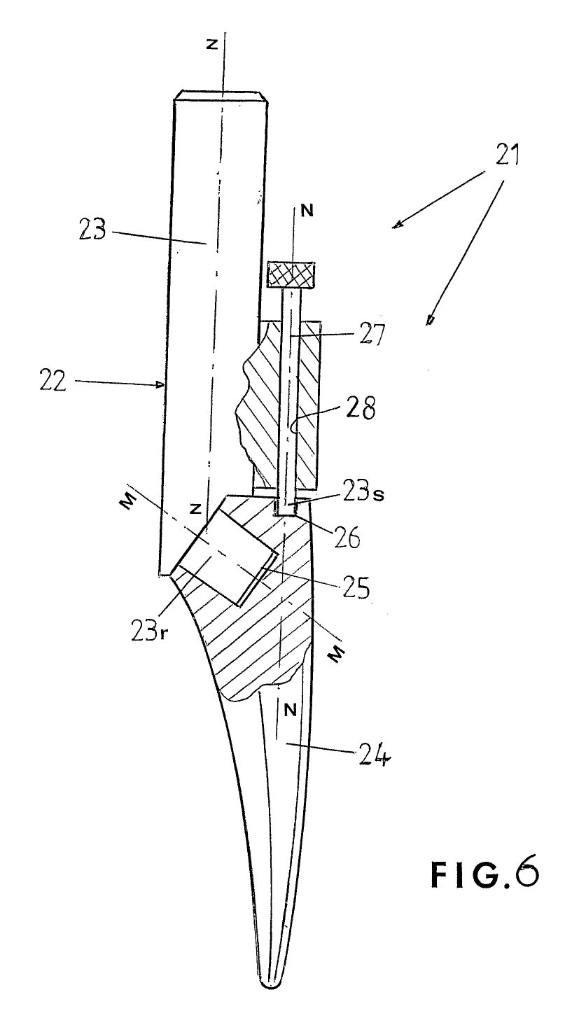

FIG. 6 is a longitudinal section through the device according to FIGS. 4, 5 with also a second terminal portion thereof engaged with the other one of the two recesses formed in the stem of the prosthesis.

Considering firstly FIG. 1 this shows how an assembly 1 according to the invention comprises, in a first example of embodiment, a rasp 2 able to be inserted in a known manner inside the intramedullary cavity 3 of a bone 4, and a device 5 able to perform insertion of the said rasp 2 inside the cavity 3 and the subsequent extraction thereof after it has performed preparation of the bone 4 for application of a prosthesis (not shown in the drawings).

In an assembly 1 according to the invention the free end 2n of the rasp 2 has, formed therein, a first recess 6 able to be joined in a complementary manner with the end of the modular neck of a prosthesis, and a second recess 7 with an axis T-T substantially intersecting the axis V-V of the said first recess. The device 5, which may be defined as an insertion/extraction device, has two terminal portions 8t, 8s which are arranged alongside each other, the first portion 8t being shaped and oriented so as to be able to be engaged in a complementary manner with the abovementioned first recess 6 and the second portion 8s being connected to means 10, 11, 12, 13, 14, fixed onto a handle 8 which will be described more fully below and which, when operated, perform insertion of said portion into the abovementioned second recess 7.

FIG. 2 shows the assembly 1 at the instant when the first terminal portion 8t (indicated by a broken line so that can be seen more clearly) has been inserted into the first recess 6, applying it using simple known methods. It can be seen moreover how a lever 10 is fixed onto the handle 8 so as to be able to rotate with respect to the fixing point P. This lever 10 has a projection 11 on which there is pivotably hinged at one end a longitudinal rod 12 terminating in the abovementioned second terminal portion 8s, all the component parts being formed so that a rotation R of the lever 10 produces a linear displacement 9 of the longitudinal rod 12 in the direction of the axis T-T of the said second cavity 7 and parallel to the axis X-X of the handle 8.

Starting from the situation shown in FIG. 2 and causing the lever 10 to perform a rotation R, in the case shown so as to move it towards the handle 8, arranging it parallel thereto, the second terminal portion 8s is displaced in linear fashion and is inserted coaxially inside the said second recess 7 as shown in FIG. 3.

So as to ensure that the second terminal portion 8s may perform the said displacement along the axis T-T of the second recess 7 and parallel to the handle 8, the lever 10 has a projection 11 on which an eyelet 13 is formed, and the said longitudinal rod 12 is pivotably hinged on this projection 11 by means of a fixing element 14 (for example a small bolt, a small rivet, etc.) which passes through the said eyelet 12 and allows the relative sliding and rotational movements of the projection 11, which rotates with the lever 10, and the longitudinal rod 12. Other similar methods, however, may be applied.

In order to keep the longitudinal rod 12 constantly parallel and adjacent to the handle 8, the inventor has envisaged applying onto the latter guide means which may also consist of a simple relief 15, as shown in the drawings, which projects from the handle 8 and extends parallel to the axis X-X of the latter and to the said axis T-T of the already described second recess 7. Other means, however, for guiding and facilitating the engagement between the second terminal portion 8s of the handle 8 and the respective recess 7 may also be applied.

These means have not been shown in the drawings.

When an assembly according to the invention is in the position shown in FIG. 3, the rasp 2 is gripped between the two terminal portions 8t, 8s of the handle 8 and, keeping the lever 10 in the position shown in the drawing, it is possible to extract the rasp 2 easily from the cavity 3 of the bone 4 by exerting a pulling force on the said handle 8.

This pulling force may also be, if necessary, of considerable magnitude, and causes the abovementioned extraction of the rasp 2 owing to the fact that the latter is gripped between the two terminal portions 8t, 8s of the handle 8, which act in the manner of proper gripping pincers.

If we then consider FIGS. 4 to 6, these show a second example of embodiment 21 of an assembly according to the invention, consisting of an insertion/extraction device 22 which, although having the same functional characteristics as that described above, differs from it with regard to the means used to cause the axially sliding of a second terminal portion thereof.

In the case in question (considering firstly FIG. 4), the device 22 also has, as in the previous example, a first terminal portion 23s and a second terminal portion 23t which project from a handle 23 integral therewith. The first terminal portion 23s has the same shape as the terminal portion of the neck which must be engaged with the stem 24 to be connected to the device 21 and has, similar to the previous case, a first recess 25 which matches the said first terminal portion 23s and a second recess 26 which is almost cylindrical and able to be engaged with the said second terminal portion 23t connected to the handle 23.

The axis Z-Z of the handle 23 is parallel to that N-N of the second recess 26.

Once the terminal portion 23s has been inserted inside the first recess 25, moving it along the longitudinal axis M-M of the latter, gripping between the insertion/extraction device 21 and the stem 24 is obtained, as already in the previous case, by moving the second terminal portion 23t along the axis N-N of the second recess 26, intersecting the said axis M-M, until it presses against its end wall 26f. In this example of embodiment, the said second terminal portion 23s is the end of a threaded element 27 screwed into a threaded through-hole 28 formed in the handle 23, and its said movement along the axis N-N is obtained by causing it to perform the number of rotations necessary for obtaining complete penetration thereof inside the second recess 26.

This second method of designing an assembly according to the invention is considered by the inventor to be simpler and low-cost and therefore more suited for ordinary cases where there is no need for engagement with particularly high gripping forces.

1-6. (canceled)

7. Assembly consisting of an insertion/extraction device and a preparatory instrument or a prosthetic part used in orthopaedic surgery operations which involve the insertion thereof inside the intramedullary cavity of a bone, wherein the free end of the said preparatory instrument or that prosthetic part has, formed therein, a first recess able to be engaged in a complementary manner with an end of a modular neck, and a second recess having an axis substantially intersecting an axis of the first recess and wherein the device is provided with a handle having its axis parallel to that of the second recess and having a first terminal portion shaped so as to engage in a complementary manner with the said recess, and a second terminal portion adjacent to the first recess and connected to means which are fixed onto the said handle and which, when they are operated, perform the coaxial insertion of said portion into the said second recess.

8. The assembly according to claim 1, wherein the said means which perform coaxial insertion and/or extraction of the said terminal portion into/from the second recess consist of a lever which is fixed onto the handle so as to be able to rotate with respect to the fixing point and has a projection on which there is pivotably hinged at one end a longitudinal rod terminating at the other end in the said second terminal portion, all the components being formed so that a rotation of the said lever produces a linear displacement of the said longitudinal rod in the direction of the said axis of the second recess.

9. The assembly according to claim 2, wherein an eyelet is formed on the said projection of the lever, and the said longitudinal rod is pivotably hinged on the said projection by means of a fixing element which passes through the above mentioned eyelet and allows the relative sliding and rotational movements of the projection and the longitudinal rod.

10. The assembly according to claim 3, wherein the handle, has mounted thereon, guide means able to keep the longitudinal rod parallel and adjacent to the said handle when the longitudinal rod is displaced as a result of a rotation of the said lever.

11. The assembly according to claim 3, wherein the said guide means consist of a relief which projects from the handle underneath the longitudinal rod and extends parallel to the handle itself and the said axis of the above mentioned second recess.

12. The assembly according to claim 1, wherein the said means which perform coaxial insertion and/or extraction of the said second terminal portion inside the said second recess consist of a threaded element screwed into a threaded through-hole formed in the handle.
